(19) United States
(12) Patent Application Publication
(10) Pub. No.: US 2008/0234619 A1
(43) Pub. Date: Sep. 25, 2008

(54) Portable Hyperthermia Apparatus

(75) Inventors: J. Michael Fausset, Lafayette, IN (US); Robert J. Schindler, Euclid, OH (US)

Correspondence Address: PEPPER HAMILTON LLP, ONE MELLON CENTER, 50TH FLOOR, 500 GRANT STREET, PITTSBURGH, PA, 15219, US

(73) Assignee: THERMAL THERAPEUTIC SYSTEMS, INC., Pittsburgh, PA (US)

(21) Appl. No.: 12/054,013

(22) Filed: Mar. 24, 2008

Related U.S. Application Data
(60) Provisional application No. 60/896,610, filed on Mar. 23, 2007.

(51) Int. Cl. A61M 1/36

(52) U.S. Cl. 604/4.01

(57) ABSTRACT

An apparatus for implementing hyperthermia may include a reservoir cartridge having an inlet and an outlet, a disposable outflow tube having a reservoir cartridge end connected to the reservoir cartridge at the outlet and an outflow catheter end connected to an outflow catheter, a disposable inflow tube having a reservoir cartridge end connected to the reservoir cartridge at the inlet and an inflow catheter end connected to an inflow catheter, a pump connected to the outflow tube, an integrated computer in communication with at least one disposable temperature sensor and at least one pressure sensor, a thermoelectric heater, proximate to the reservoir cartridge and a housing that contains the reservoir cartridge, the heater and the integrated computer.

### CROSS REFERENCE TO RELATED APPLICATIONS

This application claims the benefit of U.S. Provisional Application No. 60/896,610 filed on Mar. 23, 2007, the entirety of which is hereby incorporated by reference.

### BACKGROUND

The use of a thermal therapy device to deliver intraperitoneal hyperthermia in conjunction with surgery and/or chemotherapy has resulted in positive survival and quality of life outcomes for patients who may have otherwise had only weeks or months to live. The dramatic response is due in part to direct contact of heat or medication and heat with diseased areas. Intraperitoneal hyperthermia has proven a successful treatment for numerous ailments, including, but not limited to, cancer. Exposing affected cells to heat, therapeutic agents and/or medication has a more aggressive and profound effect on patient outcomes.

Conventional hyperthermia apparatuses utilize a passive heating system, such as a heat exchanger, to heat a fluid to be supplied to a patient. A heat exchanger is typically connected via a set of tubes to a water tank. The water tank is commonly connected to a heater that heats water in the tank. Heated tank water is pumped to the heat exchanger, which typically has two compartments, one compartment containing water and a second compartment containing a fluid to be administered to a patient. The two compartments are typically separated by a metal plate. The heated water from the tank heats the water in the first compartment of the heat exchanger. The water in the heat exchanger then heats the metal plate which in turn heats the fluid. As such, the fluid is heated by a series of heat transfers and not by a direct heat transfer to the fluid.

This indirect approach to heating fluid results in heat loss due to the numerous points of heat exchange (i.e. from the tank to the heat exchanger, from one compartment of the heat exchanger to the metal plate, from the metal plate to the other compartment and from the heat exchanger to the patient). Moreover, conventional hyperthermia apparatuses are large and weigh in excess of three-hundred pounds. This limits mobility and storage capacity, and renders the apparatus unsuitable for use outside of operating rooms.

### SUMMARY

Before the present methods are described, it is to be understood that this invention is not limited to the particular systems, methodologies or protocols described, as these may vary. It is also to be understood that the terminology used herein is for the purpose of describing particular embodiments only, and is not intended to limit the scope of the present disclosure which will be limited only by the appended claims.

In an embodiment, an apparatus for implementing hyperthermia may include a reservoir cartridge having an inlet and an outlet, a disposable outflow tube having a reservoir cartridge end connected to the reservoir cartridge at the outlet and an outflow catheter end connected to an outflow catheter, a disposable inflow tube having a reservoir cartridge end connected to the reservoir cartridge at the inlet and an inflow catheter end connected to an inflow catheter, a pump connected to the outflow tube, an integrated computer in communication with at least one disposable temperature sensor and at least one pressure sensor, a thermoelectric heater, proximate to the reservoir cartridge and a housing that contains the reservoir cartridge, the heater and the integrated computer.

In an embodiment, a system for implementing hyperthermia may include an apparatus for implementing hyperthermia and a plurality of auxiliary temperature sensors in communication with the integrated computer. The apparatus for implementing hyperthermia may include a disposable reservoir cartridge having an inlet and an outlet, a disposable outflow tube having a reservoir cartridge end connected to the reservoir cartridge at the outlet and an outflow catheter end connected to an outflow catheter, a disposable inflow tube having a reservoir cartridge end connected to the reservoir cartridge at the inlet and an inflow catheter end connected to an inflow catheter, a pump connected to the outflow tube, an integrated computer in communication with at least one disposable temperature sensor and at least one pressure sensor, a thermoelectric heater, wherein the heater is proximate to the reservoir cartridge, and a housing that contains the reservoir cartridge, the heater and the integrated computer.

A system for implementing hyperthermia may include an apparatus for implementing hyperthermia and a pressure isolator connected to an outflow tube by a first connector tube and connected to a durable pressure sensor by a second connector tube. The apparatus for implementing hyperthermia may include a disposable reservoir cartridge having an inlet and an outlet, a disposable outflow tube having a reservoir cartridge end connected to the reservoir cartridge at the outlet and an outflow catheter end connected to an outflow catheter, a disposable inflow tube having a reservoir cartridge end connected to the reservoir cartridge at the inlet and an inflow catheter end connected to an inflow catheter, a pump connected to the outflow tube, an integrated computer in communication with at least one disposable temperature sensor and at least one pressure sensor, a thermoelectric heater proximate to the reservoir cartridge, and a housing that contains the reservoir cartridge, the heater and the integrated computer, and comprises a durable pressure sensor.

### BRIEF DESCRIPTION OF THE DRAWINGS

Embodiments of the invention are better understood with reference to the following drawings. The elements of the drawings are not necessarily to scale relative to each other. Like reference numerals designate corresponding similar parts.

Aspects, features, benefits and advantages of the present invention will be apparent with regard to the following description and accompanying drawings, of which:

FIG. 1 depicts exemplary elements of a hyperthermia apparatus according to an embodiment.

FIG. 2 depicts exemplary elements of a hyperthermia apparatus operating in prime mode according to an embodiment.

FIG. 3 depicts exemplary elements of a hyperthermia apparatus contained in a housing according to an embodiment.

FIG. 4 depicts an exemplary reservoir cartridge and screen according to an embodiment.

FIG. 5 depicts an exemplary pressure isolator according to an embodiment.

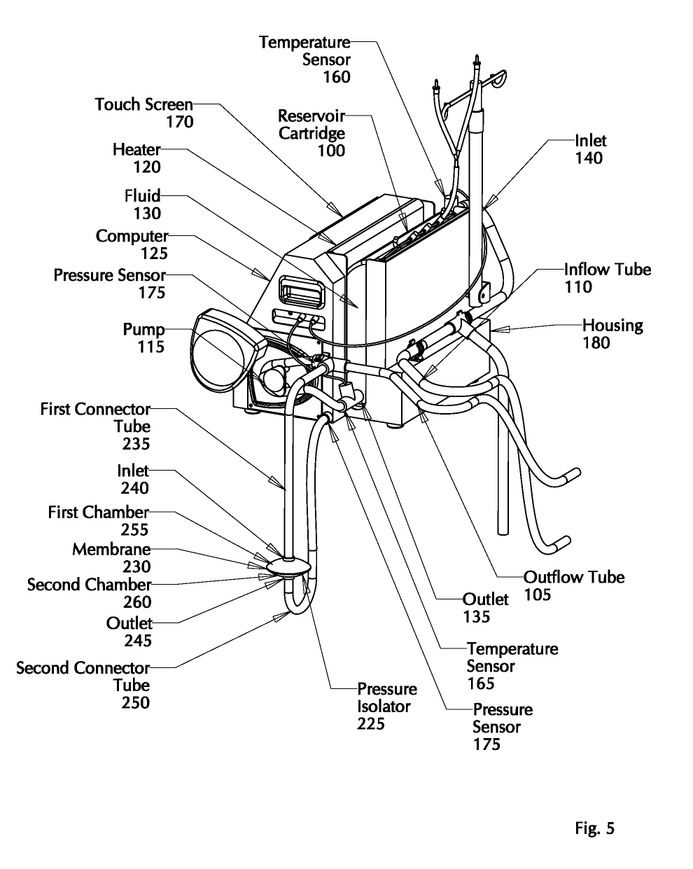

### DETAILED DESCRIPTION

It must be noted that as used herein and in the appended claims, the singular forms “a,” “an,” and “the” include plural reference unless the context clearly dictates otherwise. Thus, for example, reference to a “pump” is a reference to one or more pumps and equivalents thereof known to those skilled in the art, and so forth. Unless defined otherwise, all technical and scientific terms used herein have the same meanings as commonly understood by one of ordinary skill in the art.

As used herein, the term “comprising” means “including, but not limited to.” As used herein, the term “about” means plus or minus 10% of the numerical value of the number with which it is being used. For example about 50% means in the range of 45%-55%.

As used herein, the term “therapeutic agent” means an agent utilized to treat, combat, ameliorate or prevent an unwanted condition or disease of a patient. In an embodiment, a therapeutic agent may include a chemotherapeutic agent.

“Administering” when used in conjunction with a therapeutic agent means to administer a therapeutic agent directly into or onto a target tissue or to administer a therapeutic agent to a patient whereby the therapeutic agent positively impacts the tissue to which it is targeted. “Administering” a composition may be accomplished by oral administration, injection, infusion or absorption or in conjunction with intraperitoneal hyperthermia or by a combination of such techniques. Such techniques may further include heating, radiation and ultrasound.

The term “target”, as used herein, refers to the material for which either deactivation, rupture, disruption or destruction or preservation, maintenance, restoration or improvement of function or state is desired. For example, diseased cells, pathogens, or infectious material may be considered undesirable material in a diseased subject and may be a target for therapy.

The term “treating” may be taken to mean prophylaxis of a specific disorder, disease or condition, alleviation of the symptoms associated with a specific disorder, disease or condition and/or prevention of the symptoms associated with a specific disorder, disease or condition.

The term “patient” generally refers to any living organism to which to compounds described herein are administered and may include, but is not limited to, any non-human mammal, primate or human. Such “patients” may or my not be exhibiting the signs, symptoms or pathology of the particular diseased state.

The terms “effective” or “therapeutically effective” as used herein may refer to eliciting a biological or medicinal response in a tissue, organ, system, animal, individual or human that is being sought by a researcher, veterinarian, medical doctor or other clinician. A biological or medicinal response may include, for example, one or more of the following: (1) inhibiting a disease, condition or disorder in an individual that is experiencing or displaying the pathology or symptoms of the disease, condition or disorder or arresting further development of the pathology and/or symptoms of the disease, condition or disorder, and (2) ameliorating a disease, condition or disorder in an individual that is experiencing or exhibiting the pathology or symptoms of the disease, condition or disorder or reversing the pathology and/or symptoms experienced or exhibited by the individual.

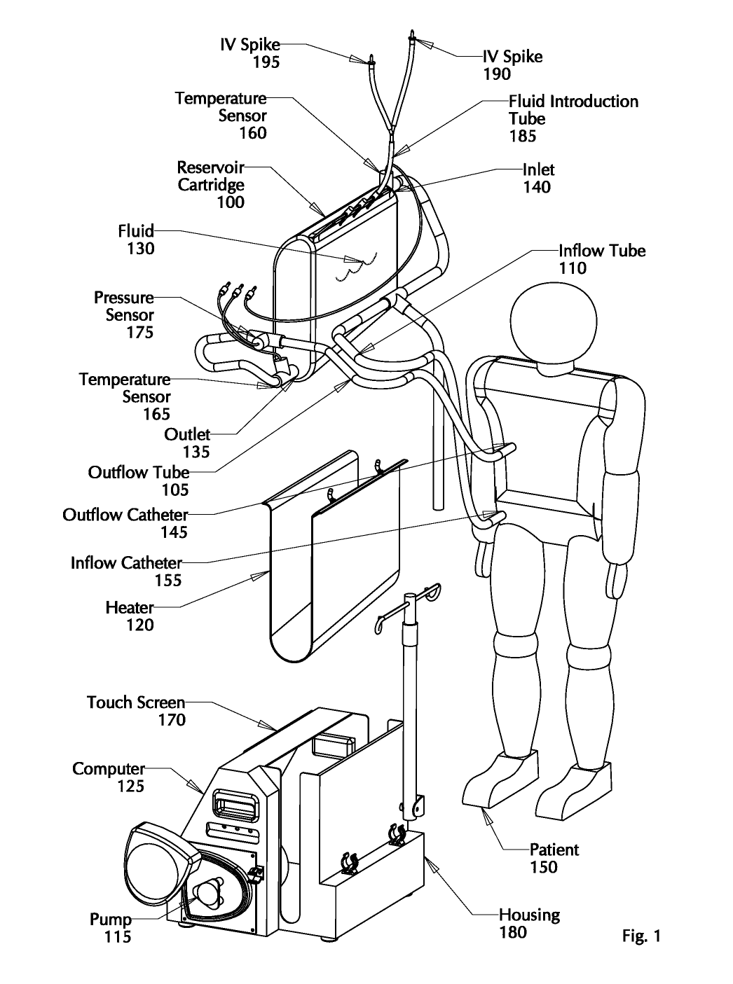

In an embodiment, a hyperthermia apparatus of the invention, as illustrated in FIG. 1, includes reservoir cartridge 100, outflow tube 105, inflow tube 110, pump 115, heater 120, computer 125 and housing 180.

In an embodiment, heater 120 may be in close proximity to reservoir cartridge 100 to maximize heat transfer. Heater 120 may be, for example, a thermoelectric heater for providing electric heat to fluid 130. A thermoelectric heater may facilitate direct heat transfer to fluid 130. In alternative embodiments, heater 120 is any type of heater known in the art, such as a water bath, immersion heater or the like.

In an embodiment, computer 125 may be an integrated computer, meaning that the computer and visual display are in the same unit. In one embodiment, the visual display may be a touch-screen. In another embodiment, computer 125 may be removable from housing 180. For example, computer 125 may be removed from housing 180 prior to transport of the apparatus, and connected to the apparatus prior to treatment of a patient.

In an embodiment, reservoir cartridge 100 stores or receives fluid 130 to be administered to a patient. Reservoir cartridge 100 may be disposable, and may have an inlet 140 and an outlet 135. In an embodiment, reservoir cartridge 100 may comprise a lock out circuit, a resistor circuit and/or the like. Lock out circuit may include a fuse link that may be deactivated after the treatment of a patient is completed. For example, a fuse link may short circuit when treatment is completed. This may prevent unauthorized use of and/or unauthorized re-use of disposable components, such as reservoir cartridge 100. Reservoir cartridge 100 may comprise one or more inlets and/or outlets. The inlets and/or outlets may be sealed to prevent the escape of fluid 130, and may facilitate the maintenance of a sterile environment when reservoir cartridge 100 is not connected to the hyperthermia apparatus.

In an embodiment, fluid 130 may be contained in reservoir cartridge 100. Fluid 130 may be introduced into reservoir cartridge 100 via fluid introduction tube 185. Fluid introduction tube 185 may include one or more valves, clamps, or inlets to allow one to introduce a physiologically compatible solution such as a drug into fluid 130 at a controlled rate. Such devices are known in the art and include, for example, IV spikes 190, 195.

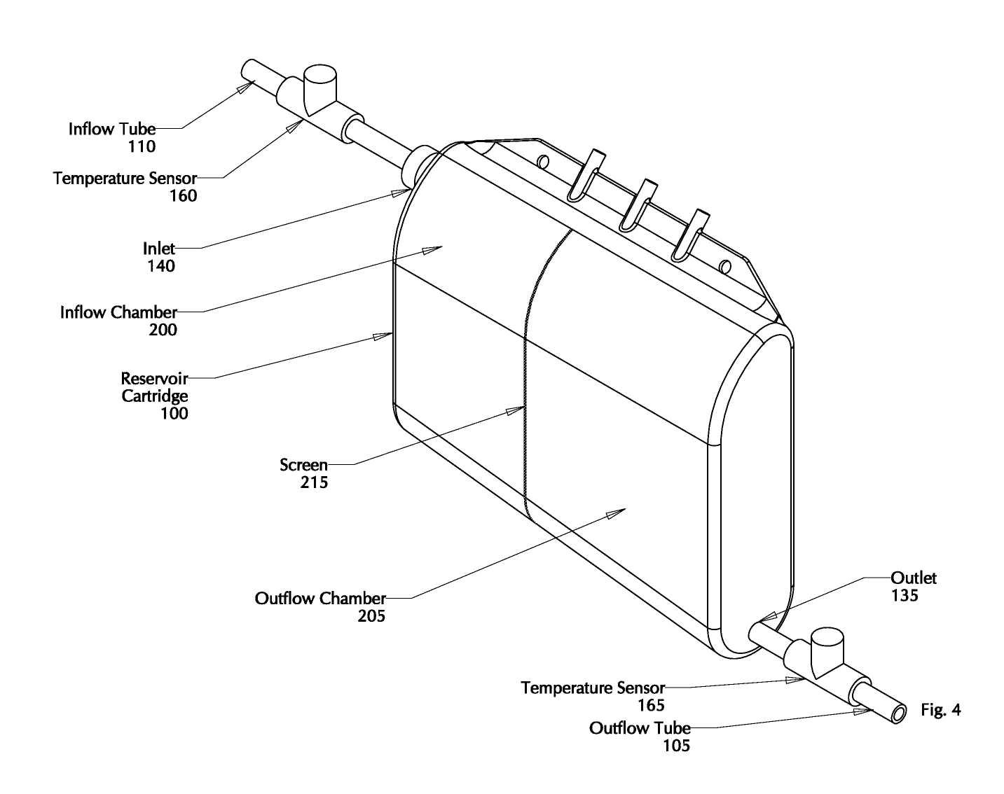

In an embodiment, reservoir cartridge 100 may be fabricated from PVC plastic film and/or other plastic materials. Reservoir cartridge 100 may be RF welded and/or RF heat sealed. In an embodiment, reservoir cartridge 100 may comprise a screen 215. FIG. 4 illustrates an exemplary reservoir cartridge 100 and screen 215. Screen 215 may be located in the proximity of inlet 140. In an embodiment, screen 215 may divide reservoir cartridge 100 into an inflow chamber 200 and an outflow chamber 205. Inflow chamber 200 may receive fluid 130 from inflow tube 110. Fluid 130 may pass through screen 215 into second chamber 205. Screen 215 may filter macroscopic residue, such as fatty tissue, from fluid 130 returning to reservoir cartridge 100 via inflow tube 110. Screen 215 may be fabricated from plastic and/or the like and may be disposable. Screen 215 may be able to filter particles having a size of about 100-140 microns or larger.

In an embodiment, fluid 130 comprises a sterile fluid. In another embodiment, fluid 130 comprises drugs, medication or the like. In an embodiment, hyperthermia assists to render a chemotherapeutic agent more effective against a target disease than the agent would be without the use of hyperthermia. In an embodiment, fluid 130 may comprise one or more chemotherapeutic agents such as cyclophosphamide, doxorubicin, melphalan, mitomycin C, cisplatin, gemcitabine, mitoxantrone, oxaliplatin, etoposide, irinotecan, paclitaxel, docetaxel, 5-Fluorouracil, floxuridine, carboplatin, or other chemotherapeutic agents as would be well-known by one of skill in the art.

In an embodiment, reservoir cartridge 100 and fluid 130 are heated by heater 120. Because heater 120 may be utilized to maximize heat transfer, the apparatus' power consumption may be up to about 15 amps. Alternatively, other power consumption values may be up to about 30 amps.

In an embodiment, once fluid 130 reaches a desired temperature, it may be pumped through outflow tube 105 to patient 150 via pump 115. In an embodiment, outflow tube 105 is disposable, with a proximal end and a distal end. Outflow tube 105 is connected at its proximal end (reservoir cartridge end) to reservoir cartridge 100 at outlet 135, while the distal end (outflow catheter end) of outflow tube 105 is connected to outflow catheter 145. Outflow catheter 145 may be inserted into a patient 150.

In an embodiment, pump 115 may be a paddle wheel, a roller pump, a pulsatile pump, centrifugal pump and/or the like. In an embodiment, pump 115 is in contact with outflow tube 105, and pumps fluid 130 at a rate of up to about 4,000 ml per minute. The high flow rate as compared to prior devices may be critical in providing beneficial treatment by maximizing contact of fluid with a patient. In addition, a high flow rate maintains the temperature of the fluid, which is an important feature of effective hyperthermia. The flow rate in combination with heat provided by a fluid may thereby increase the efficacy of a hyperthermia treatment.

In an embodiment, fluid 130 is administered to a patient 150, and is then re-circulated to reservoir cartridge 100 through inflow tube 110. Re-circulation of the heated fluid may be used to elevate a patient's core temperature and/or to maintain an elevated temperature for a period of time.

In an embodiment, inflow tube 110 is disposable, with proximal and distal ends. Inflow tube 110 may be connected at its distal end (inflow catheter end) to a patient 150 via inflow catheter 155, and may be connected at its proximal end (reservoir cartridge end) to reservoir cartridge 100 at inlet 140. Fluid 130 coming from patient 150 may be re-heated in reservoir cartridge 100, and once again pumped to a patient 150. This process may continue for a specified period of time with multiple cycles of re-circulation, as may be desired for a given therapeutic effect.

In an embodiment, one or more of inflow tube 110 and outflow tube 105 may comprise a flange or other similar portion. Inflow tube 110 may have a flange at its distal end (inflow catheter end) and outflow tube 105 may have a flange at its distal end (outflow catheter end). The flange may be fabricated from plastic and/or any other suitable material. In an embodiment, the flange may assist a physician or other healthcare professional to more quickly and efficiently suture the inflow tube 110 and/or the outflow tube 105 to the patient 150. Moreover, the flange may serve as a seal for the distal end (inflow catheter end) of the inflow tube 110 and/or the distal end (outflow catheter end) of the outflow tube 105.

According to an embodiment, the hyperthermia apparatus of the invention includes sensors for monitoring the temperature and pressure of the fluid. In an embodiment, a temperature sensor may be a standard thermistor, an infrared thermistor or the like. As illustrated in FIG. 1, temperature sensors 160, 165 may be located in reservoir cartridge 100 at the proximal ends of inflow tube 110 and outflow tube 105. Temperature sensors 160, 165 may allow monitoring of a fluid's 130 temperature as it both enters and leaves reservoir cartridge 100. In an embodiment, temperature sensors 160, 165 are disposable. In an embodiment, temperature sensors 160, 165 are in communication with a computer 125.

In an embodiment, a system for implementing hyperthermia may include one or more auxiliary temperature sensors and a hyperthermia apparatus such as that described in this disclosure. The auxiliary temperature sensors may be placed on and/or in the patient at various locations. One or more auxiliary temperature sensors may be plug-in thermistors that may be connected to the hyperthermia apparatus via a standard connection or the like. In another embodiment, one or more auxiliary temperature sensors may communicate with the hyperthermia apparatus wirelessly. In an embodiment, a healthcare professional may select one of the auxiliary temperature sensors to use as a reference. The healthcare professional may be able to monitor the temperature at the location of the auxiliary temperature sensors, and the hyperthermia apparatus may control the temperature of the fluid based on the selected auxiliary temperature sensor rather than the temperature sensors 160, 165 located in the hyperthermia apparatus.

As illustrated in FIG. 1, pressure sensor 175 is located, according to an embodiment, in outflow tube 105 near reservoir cartridge 100. Preferably, pressure sensor 175 is located within outflow tube 105 downstream from pump 115. In an embodiment, pressure sensor 175 is located within outflow tube 105 immediately downstream from pump 115. Pressure sensor 175 may allow monitoring of fluid's 130 pressure as fluid 130 leaves reservoir cartridge 100. In an embodiment, pressure sensor 175 is in communication with computer 125. In an embodiment, pressure sensor 175 is disposable.

In another embodiment, housing may contain a pressure sensor 220. Pressure sensor 220 may be durable. Pressure sensor 220 may measure pressure of fluid 130 via a pressure isolator 225. Pressure isolator 225 may comprise a first chamber 255, a second chamber 260, an inlet 240, an outlet 245 and/or a membrane 230. A first connector tube 235 may connect outflow tube 105 to inlet 240. A second connector tube 250 may connect outlet 245 to pressure sensor 220. Membrane 230 may separate first chamber 255 from second chamber 255. Membrane 230 may be fluid impermeable and may prevent fluid 130 from coming into contact with pressure sensor 220. In an embodiment, pressure sensor 220 may measure the pressure of fluid 130 based on a pressure differential between first chamber 255 and second chamber 260.

Although the figures illustrates specific placements of temperature sensors 160, 165 and pressure sensors 175, 220 it is understood that sensors 160, 165, 175, 220 may be placed in different locations on the apparatus. In addition, one or more of temperature sensors 160, 165 and pressure sensors 175, 220 may wirelessly communicate with computer 125. Moreover, additional temperature and/or pressure sensors may be implemented by the device of the invention.

The apparatus of the invention may be used to monitor a fluid's 130 temperature while the fluid is in reservoir cartridge 100, which may allow for accurate temperature control to within about plus or minus one-half of one degree Centigrade (0.5° C.). In other words, the temperature variation of a fluid from when it leaves reservoir cartridge 100 to when it enters a patient 150 may be, for example, about 0.5° C. or less. In a preferred embodiment, the temperature of fluid 130 in reservoir cartridge 100 does not exceed about 43° C. As such, the temperature of fluid 130 when administered to a patient 150 preferably does not exceed about 42.5° C. The temperature of a fluid 130 in reservoir cartridge 100 may be maintained at a temperature other than 43° C., for example, any temperature desired by an operator to achieve the desired therapeutic effect, or for operation of the device in prime mode, as described below.

Computer 125 and touch screen 170 are, in one embodiment, configured to provide audible and visible alarms if certain conditions occur. For example, if the temperature of a fluid 130 in reservoir cartridge 100 exceeds a specified temperature, a visible and/or audible alarm may be triggered. Likewise, if the pressure or temperature of fluid 130 exceeds a preset threshold, a visible and/or audible alarm may be triggered.

As a safety precaution, heater 120, according to one embodiment, stops providing heat if a fluid's 130 temperature exceeds a specified temperature. In an embodiment, heater 120 stops providing heat if fluid's 130 pressure exceeds a specified level.

Similarly, pump 115, in an embodiment, stops pumping fluid 130 if the fluid's 130 pressure exceeds a specified level. In an embodiment, pump 115 stops pumping fluid 130 if fluid's 130 temperature exceeds a specified level.

Computer 125 may include a processor and a processor-readable storage medium. Computer 125 is programmable and capable of receiving input from a user. For example, a user may specify temperature levels, pressure levels or the like via the touch screen 170 or other input interfaces. A user may also input other information, such as the duration of the treatment, the amount of time the apparatus is to operate in prime mode or the like. In an embodiment, computer 125 may record data such as measurements associated with treatment and the like. For example, during the treatment of a patient, computer 125 may record one or more temperatures at one or more temperature sensors 160, 165, auxiliary temperature sensors and/or the like. Computer 125 may also record flow rates, pressure values, treatment time and/or the like.

Computer 125 is in communication with pump 115, heater 120, temperature sensors 160, 165, and pressure sensor 175. Computer 125 controls the operation of pump 115 and monitors temperature sensors 160, 165 and pressure sensor 175. In an embodiment, if the temperature or pressure of fluid 130 exceeds a specified level, computer 125 provides audible and visual alarm signals. In another embodiment, computer 125 shuts off heater 120 if fluid's 130 temperature exceeds a specified temperature or if fluid's 130 temperature is outside a specified range of temperatures. Likewise, in an embodiment, computer 125 shuts off pump 115 if fluid's 130 pressure exceeds a specified pressure level or if fluid's 130 pressure is outside a specified range of pressure levels. Computer 125 may shut off pump 115 if fluid's 130 temperature exceeds a specified temperature or if fluid's 130 temperature is outside a specified range of temperatures. Similarly, computer 125 may shut off heater 120 if fluid's 130 pressure level exceeds a specified pressure level or if fluid's 130 pressure level is outside a specified range of pressure levels.

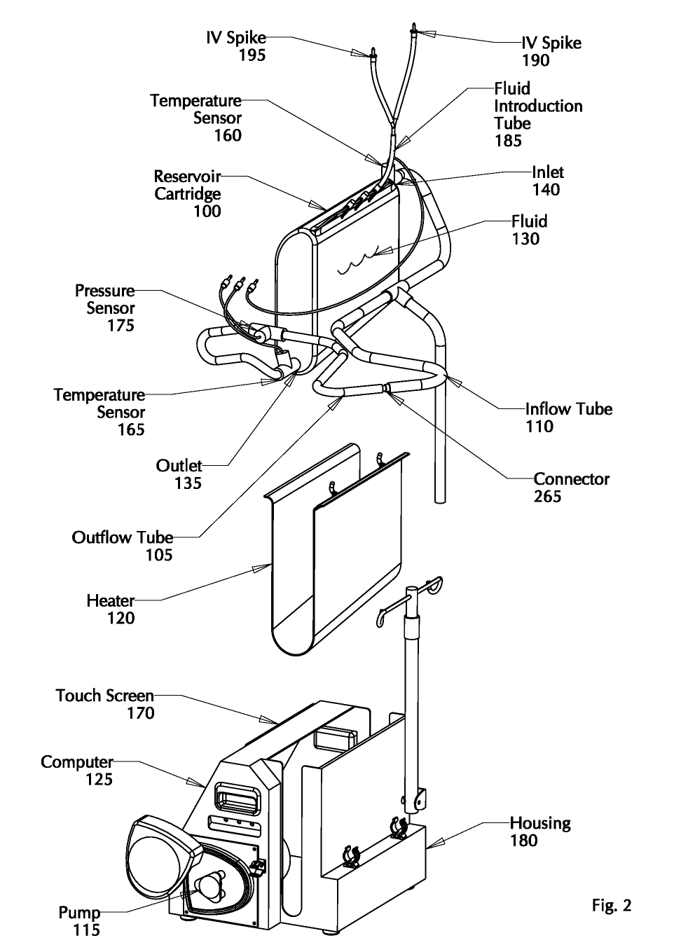

In an alternate embodiment, the apparatus of the invention operates without being connected to a patient. This is referred to as “prime mode” and is illustrated in FIG. 2. In prime mode, the apparatus prepares fluid 130 to be administered to a patient by heating and re-circulating fluid 130. In prime mode, outflow tube 105 may be connected to the inflow tube 110 via connector 265. A variety of tubing connectors suitable for use in the invention are known in the art and may include, for example, a barbed tubing connector or the like, or other connector as may be suitable to achieve the desired connector function. As such, the fluid 130 may be pumped from reservoir cartridge 100 through outflow tube 105 through connector 265 and back to reservoir cartridge 100 through inflow tube 110. Fluid 130 may be pumped for a specified period of time before fluid 130 is administered to a patient. When operating in prime mode, the temperature of fluid 130 in the reservoir may be maintained at a temperature up to, for example, 53° C. The device of the invention may be run in prime mode to ensure that the temperature of fluid 130 does not drop below an acceptable level before the apparatus is connected to a patient. An operator may set a time period for the apparatus to operate in prime mode.

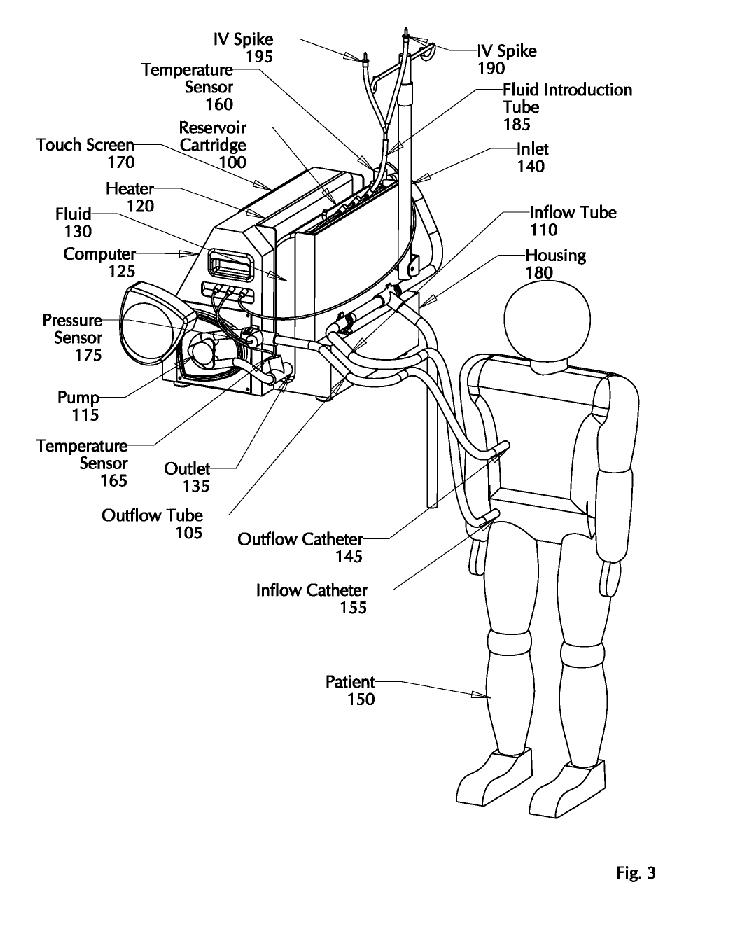

In an embodiment, reservoir cartridge 100, heater 120 and computer 125 are contained in housing 180 (FIG. 3). In one embodiment, these elements are located in close proximity to each other. The proximity of elements contribute to the apparatus' portability and ease of use in a variety of clinical settings, including both inside and outside of an operating room.

It will be appreciated that various of the above-disclosed and other features and functions, or alternatives thereof, may be desirably combined into many other different systems or applications. Various presently unforeseen or unanticipated alternatives, modifications, variations or improvements therein may be subsequently made by those skilled in the art which are also intended to be encompassed by the following claims.

What is claimed is:

1. An apparatus for implementing hyperthermia comprising:
a reservoir cartridge having an inlet and an outlet;
a disposable outflow tube having a reservoir cartridge end connected to the reservoir cartridge at the outlet and an outflow catheter end connected to an outflow catheter;
a disposable inflow tube having a reservoir cartridge end connected to the reservoir cartridge at the inlet and an inflow catheter end connected to an inflow catheter;
a pump connected to the outflow tube;
an integrated computer in communication with at least one disposable temperature sensor and at least one pressure sensor;
a thermoelectric heater, wherein the heater is proximate to the reservoir cartridge; and
a housing that contains the reservoir cartridge, the heater and the integrated computer.

2. The apparatus according to claim 1, wherein the reservoir cartridge contains sterile fluid.

3. The apparatus according to claim 1, wherein the reservoir cartridge is disposable.

4. The apparatus of claim 1, wherein the integrated computer comprises an integrated touch screen.

5. The apparatus according to claim 1, wherein the integrated computer is removable from the housing.

6. The apparatus according to claim 1, wherein the reservoir cartridge comprises a disposable temperature sensor located at the reservoir cartridge end of the inflow tube.

7. The apparatus according to claim 1, wherein the reservoir cartridge comprises a disposable temperature sensor located at the reservoir cartridge end of the outflow tube.

8. The apparatus according to claim 1, wherein the outflow tube comprises a disposable pressure sensor located at the outflow catheter end.

9. The apparatus according to claim 1, wherein the reservoir cartridge is configured to:
comprise a fluid, and
maintain the fluid's temperature at 43 degrees Centigrade or lower.

10. The apparatus according to claim 1, wherein the apparatus is configured to maintain a fluid's temperature at 42.5 degrees Centigrade or lower when the fluid is administered to a patient.

11. The apparatus according to claim 1, wherein the apparatus is configured to maintain a temperature difference between the temperature of a fluid in the reservoir cartridge and the temperature of the fluid when administered to a patient of no more than ±0.5 degrees Centigrade.

12. The apparatus according to claim 1, wherein the apparatus is configured to maintain a fluid's temperature at 53 degrees Centigrade or lower when the apparatus is operating in prime mode.

13. The apparatus according to claim 1, wherein the apparatus further comprises at least one temperature alarm for providing a signal when a fluid's temperature exceeds a specified temperature.

14. The apparatus according to claim 13, wherein the signal is an audible signal.

15. The apparatus according to claim 13, wherein the signal is a visual signal.

16. The apparatus according to claim 13, wherein the heater is configured to stop providing heat when the fluid's temperature exceeds a specified temperature.

17. The apparatus according to claim 1, wherein the apparatus further comprises at least one pressure alarm for providing a signal when a fluid's pressure exceeds a specified pressure level.

18. The apparatus according to claim 17, wherein the signal is an audible signal.

19. The apparatus according to claim 17, wherein the signal is a visual signal.

20. The apparatus according to claim 17, wherein the pump is configured to stop pumping the fluid until the fluid's pressure level is less than or equal to the specified pressure level.

21. The apparatus according to claim 1, wherein the pump is configured to pump a fluid at approximately 4000 ml. per minute.

22. The apparatus according to claim 1, wherein the apparatus' power consumption is approximately 15 amps.

23. The apparatus according to claim 1, further comprising a lock out circuit configured to short circuit when treatment is concluded.

24. The apparatus according to claim 1, wherein the reservoir cartridge comprises:
a fluid; and
a screen, wherein the screen divides the reservoir cartridge into an inflow chamber and a an outflow chamber, wherein the inflow chamber is connected to the inflow tube, wherein the outflow chamber is connected to the outflow tube.

25. A system for implementing hyperthermia comprising:
an apparatus for implementing hyperthermia comprising:
a disposable reservoir cartridge having an inlet and an outlet,
a disposable outflow tube having a reservoir cartridge end connected to the reservoir cartridge at the outlet and an outflow catheter end connected to an outflow catheter,
a disposable inflow tube having a reservoir cartridge end connected to the reservoir cartridge at the inlet and an inflow catheter end connected to an inflow catheter,
a pump connected to the outflow tube,
an integrated computer in communication with at least one disposable temperature sensor and at least one pressure sensor,
a thermoelectric heater, wherein the heater is proximate to the reservoir cartridge, and
a housing that contains the reservoir cartridge, the heater and the integrated computer; and
a plurality of auxiliary temperature sensors in communication with the integrated computer.

26. The system of claim 25, wherein the integrated computer is configured to:
receive, from a user, a selection of one of the plurality of auxiliary temperature sensors; and
control a temperature of a fluid based on one or more measurements associated with the selected auxiliary temperature sensor.

27. A system for implementing hyperthermia, the system comprising:
an apparatus for implementing hyperthermia, the apparatus comprising:
an apparatus for implementing hyperthermia comprising:
a disposable reservoir cartridge having an inlet and an outlet,
a disposable outflow tube having a reservoir cartridge end connected to the reservoir cartridge at the outlet and an outflow catheter end connected to an outflow catheter,
a disposable inflow tube having a reservoir cartridge end connected to the reservoir cartridge at the inlet and an inflow catheter end connected to an inflow catheter,
a pump connected to the outflow tube,
an integrated computer in communication with at least one disposable temperature sensor and at least one pressure sensor,
a thermoelectric heater, wherein the heater is proximate to the reservoir cartridge, and
a housing that contains the reservoir cartridge, the heater and the integrated computer, wherein the housing comprises a durable pressure sensor; and
a pressure isolator, wherein the pressure isolator is connected to the outflow tube by a first connector tube, wherein the pressure isolator is connected to the durable pressure sensor by a second connector tube.

28. The system of claim 27, wherein the pressure isolator comprises:
an inlet;
an outlet;
a first chamber connected to the inlet of the pressure isolator;
a second chamber connected to the outlet of the pressure isolator;
a membrane, wherein the membrane separates the first chamber and the second chamber,
wherein the durable pressure sensor is configured to measure a pressure of a fluid based on a pressure differential between the first chamber and the second chamber.
